(19) United States
(12) Patent Application Publication
(10) Pub. No.: US 2005/0227949 A1
(43) Pub. Date: Oct. 13, 2005

(54) Compositions and methods for treatment of viral and bacterial infections

(76) Inventor: Nasrola Edalatpour, Newport Beach, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/820,355

(22) Filed: Apr. 7, 2004

(51) Int. Cl. A61K 31/60; A61K 31/4439; A61K 31/415; A61K 31/192

(52) U.S. Cl. 514/165; 514/406; 514/338; 514/569; 514/570

(57) ABSTRACT

Compositions and methods are presented that are useful for treatment of viral and/or bacterial infections, and especially the common cold or flu. Particularly preferred combinations include those orally administered, preferably early in the course of the symptoms of the cold or flu, and comprising (a) an H2-histamine receptor antagonist and (b) acetaminophen, NSAID, or other anti-pyretic, analgesic agent.

### FIELD OF THE INVENTION

The field of the invention is pharmaceutical compositions and methods for treatment of viral and bacterial infections, and especially treatment of symptoms associated with the viral and bacterial cold.

### BACKGROUND OF THE INVENTION

Vaccinations are commonly used to induce immunity against a variety of diseases in mammalians. However, most vaccinations are limited in their efficacy where the bacteria or viruses change their immunogenic epitopes to evade recognition and destruction by the immune system. Among other examples, influenza viruses are representative members of such pathogens that are difficult to treat and/or eradicate using vaccinations. To improve treatment of diseases caused by immunologically evasive pathogens, or more commonly where there is no or inadequate prior specific immunization, antiviral or antibacterial agents may be employed to interfere with the pathogen's reproduction or other step in the life cycle.

For example, there are numerous antiviral agents known in the art that reduce replication in a host cell (e.g., via inhibition of a reverse transcriptase or other viral polymerase), and at least some of these agents are reported as being effective in reducing viral titers in vitro. However, effective in vivo treatment is often hampered by low tolerance of the patient towards the antiviral drug and relatively fast development of resistance. Still further, most antiviral drugs are typically selective towards a specific virus or virus family, and are often ineffective against other viruses. For example, while several antiviral agents are relatively effective against retroviruses, such agents are typically entirely ineffective in treatment of influenza viruses.

Similarly, numerous antibiotics are known in the art. Fortunately, most of the antibiotics are effective against a relatively broad spectrum of bacteria, and if administered properly, will provide effective antibacterial treatment. However, use of almost all of the known antibiotics is also associated with significant adverse effects. Among other things, resistance often develops relatively quickly, thus precluding future use of that antibiotic for that disease. Worse yet, as antibiotic resistance if often genetically determined and bacteria frequently exchange genetic information, a previously developed resistance against an antibiotic in treatment of a first disease may be transferred to bacteria associated with a second disease.

Unfortunately, the above limitations of antiviral and antibacterial agents are especially evident in the case of viral and bacterial colds, which have a relatively high rate of recurrence and persistence. Moreover, in most patients afflicted with bacterial or viral cold, unbalanced immune response and other adaptive mechanisms of the patient often significantly contribute to the symptomatic discomfort.

For example, typical unbalanced immune responses associated with common colds are related to an exaggerated histamine response, which manifests itself as sneezing, partial airway constriction, sore throat, overproduction of mucopolysaccharides, inflammatory reaction of various tissues, etc. Consequently, various cold medicines include H1-Histamine receptor antagonists (e.g., ethanolamines, ethylenediamines, piperidines, piperazines, alkylamines, phenothiazines, etc.) to at least blunt the histamine response. However, many H1-Histamine receptor antagonists, and especially the first generation antagonists, tend to exhibit anticholinergic effects, while second generation H1-Histamine receptor antagonists often inhibit potassium rectifier currents (and with that often effect slow repolarization).

Typical adaptive mechanisms associated with common colds may include fever, and various antipyretics are frequently used in commercially available cold medicines for reduction of fever. For example, acetaminophen or ibuprofen are known to significantly reduce fever during a cold. However, while fever reduction is often desirable, most antipyretics are ineffective against other symptoms associated with the common cold. Combinations or H1-antagonists and antipyretics are therefore often found to provide enhanced therapeutic effect. However, while such combinations at least somewhat diminish cold symptoms, numerous disadvantages remain. Most significantly, and despite their portrayal in most advertisements as removing all of the symptoms quickly and in a sustained manner, presently available combination formulations often reduce symptoms, but fail to provide satisfactory relief in most cases.

Therefore, while there are numerous compositions and methods known in the art, all or almost all of them suffer from one or more disadvantage. Consequently, there is still a need to provide improved compositions and methods for treatment of viral and bacterial colds, and especially for treatment of symptoms associated with viral and bacterial colds.

### SUMMARY OF THE INVENTION

The present invention is directed to the surprising discovery that a combination of an H2-histamine receptor antagonist and an anti-pyretic, analgesic agent are effective in treatment of various infectious diseases, and especially provide substantial symptomatic relief for patients suffering from bacterial or viral cold. Contemplated anti-pyretic, analgesic agents include (a) acetaminophen and (b) a non-steroidal anti-inflammatory agent (NSAID).

In one aspect of the inventive subject matter, a pharmaceutical kit includes at least one of an H2-histamine receptor antagonist and an anti-pyretic, analgesic agent, and an instruction to administer the antagonist in combination with the analgesic to treat at least one of a viral and bacterial cold.

Consequently, in another aspect of the inventive subject matter, a method of providing a health care product will include one step in which at least one of an H2-histamine receptor antagonist and an anti-pyretic, analgesic agent are provided. In another step, an instruction is provided to administer the H2-histamine receptor antagonist in combination with the anti-pyretic, analgesic agent to treat a viral and/or bacterial cold.

Further contemplated methods include methods of providing an over-the-counter health care product in which in one step an H2-histamine receptor antagonist is provided. In another step, the H2-histamine receptor antagonist is offered for sale in a sales display in a position proximal to a position in which an analgesic or a cold or flu medicine is offered for sale.

In a still further aspect of the inventive subject matter, a method of advertising a health care product includes one step in which sale of at least one of an H2-histamine receptor antagonist and an anti-pyretic, analgesic agent is advertised. In a further step, information is provided that a bacterial and/or viral cold can be treated with a combination of the H2-histamine receptor antagonist and the anti-pyretic, analgesic agent.

It is especially preferred that the H2-histamine receptor antagonist and acetaminophen or another anti-pyretic, analgesic agent is included in an over-the-counter product. Particularly preferred H2-histamine receptor antagonist include cimetidine, famotidine, ranitidine hydrochloride, omeprazole, and esomeprazole, while preferred anti-pyretic, analgesic agents include acetaminophen, ibuprofen, naproxen, acetyl salicylic acid, celecoxib, and rofecoxib. Most preferably, the anti-pyretic, analgesic agent and the H2-histamine receptor antagonist are in orally administrable form. While not limiting the inventive concept presented herein, it is important that the contemplated combinations be used in dosages that are substantially non-toxic, and therefore it is generally preferred that such be used within listed therapeutic ranges. For some drugs, the H2-histamine receptor antagonist can be orally administered using multiple doses of up to 2400 mg daily total, and the anti-pyretic, analgesic agent is orally administered using multiple doses of up to 3000 mg daily total. The most preferred protocol is three doses of 400 mg cimetidine every 12 hours, along with 500 mg acetaminophen at the same time. The drugs are preferably taken with a full glass of water with or without food. The combination may be taken for longer periods of time, or using a different protocol, but those other protocols may well not be any more effective.

Various objects, features, aspects and advantages of the present invention will become more apparent from the following detailed description of preferred embodiments of the invention.

### DETAILED DESCRIPTION

The inventor unexpectedly discovered that viral and bacterial infections can be treated using a combination of an H2-histamine receptor antagonist and an anti-pyretic, analgesic agent (NSAID), and that such combinations are especially useful for treatment of symptoms associated with a bacterial or viral cold or flu.

The use of H1-histamine receptor antagonists in combination with NSAIDs is well known in the art for treatment of symptoms associated with viral and bacterial cold or flu. Among other things, H1 antagonists are thought to moderate the allergic response by blocking histamine action on post-capillary venule smooth muscles, resulting in decreased vascular permeability, exudation and edema. H1-antagonists also block the action of histamine on the H1 receptors on c-type nociceptive nerve fibers, resulting in decreased itching and sneezing.

In contrast, use of H2-histamine receptor antagonists, and especially in combination with NSAIDs are especially surprising as H2-histamine receptors are primarily involved in control of basal and nocturnal gastric acid secretion (H2-histamine receptors are most commonly found on the parietal cells). Furthermore, H2-histamine receptor antagonists are highly selective in their binding to the H2-histamine receptor (with typically less than 5%, and even more typically less than 1% of H2-antagonist binding to an H1-receptor at effective H2-concentration). Therefore, in light of the known use of an H2-antagonist as an antacid, and the known high selectivity of the H2-antagonists towards the H2-receptor (and not the H1-receptor), a person of ordinary skill in the art would clearly not be motivated to use an H2-antagonist for treatment of a bacterial or viral infection, and especially not for symptomatic relief of bacterial or viral cold or flu.

The term “viral cold or flu” as used herein refers to a viral infection that produces at least one the following symptoms for at least 12 hours: cough, general or localized pain, light sensitivity, sore throat, sneezing, partial airway constriction, sometimes fever, and inflammatory reaction of various tissues (especially of mucous membranes). In many cases, viral colds are caused by influenza viruses, rhinoviruses, adenoviruses, coxsackieviruses, corona viruses, parainfluenza, and respiratory syncytial viruses. The term “bacterial cold or flu” as used herein refers to a bacterial infection that produces at least one the following symptoms for at least 12 hours: Cough, sore throat, sneezing, partial airway constriction, moderate fever (typically between 99.5° F. and 102° F.), and inflammatory reaction of various tissues (especially of mucous membranes). In many cases, bacterial colds are secondary infections and are often caused by various streptococci and pneumococci. In some cases, the terms “bacterial cold” and “viral cold” are used interchangeably to refer to a plurality of cold-associated symptoms, and are then also referred to as “common cold”.

In one especially preferred aspect of the inventive subject matter, a person having a viral cold is treated at the onset of symptoms with a combination of multiple daily doses of cimetidine (preferably up to 5 doses at 200 mg per dose) and non-time released acetaminophen (preferably up to 5 doses at 200 mg per dose) for a duration of about 1-5 days. Most preferably, cimetidine and acetaminophen are administered orally together using over-the-counter products (e.g., TAGAMET™ for cimetidine and Tylenol™ for acetaminophen). The most preferred protocol is three doses of 400 mg cimetidine every 12 hours, along with 500 mg acetaminophen at the same time. The drugs are preferably taken with a full glass of water with or without food. The combination may be taken for longer periods of time, or using a different protocol, but those other protocols may well not be any more effective.

Of course, it should be appreciated that numerous H2-antagonists other than cimetidine may be employed, and suitable alternative H2-antagonists include famotidine (e.g., as PEPCID AC™), ranitidine hydrochloride (e.g., as ZANTAC™), omeprazole (e.g., as PRILOSEC™), or esomeprazole (e.g., as NEXIUM™). Similarly, while acetaminophen is preferred, a less preferred combination with NSAIDs could include ibuprofen, naproxen (e.g., as NAPROSYN™, or ALEVE™), acetyl salicylic acid (e.g., as ASPIRIN™), celecoxib (e.g., as CELEBREX™), and rofecoxib (e.g., as VIOXX™). Still further other contemplated agents include prostaglandin reducing agents such as ketoprofen, piroxicam, sulindac, choline subsalicylate, diflunisal, indomethacin, meclofenamate, salsalate, and tolmetin.

It is generally contemplated that the compounds according to the inventive subject matter will be formulated for administration to a mammal, and especially to a human with a condition that is responsive to the administration of such compounds. Therefore, where contemplated compounds are administered in a pharmacological composition, it is contemplated that contemplated compounds can be formulated in admixture with a pharmaceutically acceptable carrier. For example, contemplated compounds can be administered orally as pharmacologically acceptable salts, or intravenously in a physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

In certain pharmaceutical dosage forms, prodrug forms of contemplated compounds may be formed for various purposes, including reduction of toxicity, increasing the organ or target cell specificity, etc. Among various prodrug forms, acylated (acetylated or other) derivatives, pyridine esters and various salt forms of the present compounds are preferred. One of ordinary skill in the art will recognize how to readily modify the present compounds to prodrug forms to facilitate delivery of active compounds to a target site within the host organism or patient. One of ordinary skill in the art will also take advantage of favorable pharmacokinetic parameters of the prodrug forms, where applicable, in delivering the present compounds to a targeted site within the host organism or patient to maximize the intended effect of the compound. Similarly, it should be appreciated that contemplated compounds may also be metabolized to their biologically active form (e.g., via hydroxylation, glycolsylation, oxidation etc.), and all metabolites of the compounds herein are therefore specifically contemplated. In addition, contemplated compounds (and combinations thereof) may be administered in combination with yet further antiviral and/or antibacterial agents. Suitable additional drugs therefore include various antibiotics (e.g., beta-lactam antibiotics, tetracycline antibiotics, oxazine antibiotics, etc.), various antiviral compounds (e.g., polymerase inhibitors), and/or compounds that stimulate the immune system.

With respect to the administration schedule, it should be recognized that contemplated compounds may be administered together, or separately. Similarly, the dosage administered will at least in part depend on the type of compound used as well as on the severity of the bacterial or viral infection. However, it is generally preferred that the compounds are co-administered at a daily dosage of less than 2000 mg total per compound, more preferably less than 1500 mg total per compound (e.g., up to 1500 mg or up to 1000 mg), and most preferably less than 1000 mg total per compound. Similarly, while it is preferred that the compounds of the inventive subject matter are orally administered in a solid dosage form (e.g., tablet, capsule, or dragee), liquid forms, and parenteral forms are also specifically contemplated.

Therefore, a pharmaceutical kit may comprise at least one of an H2-histamine receptor antagonist and an anti-pyretic, analgesic agent, and an instruction to administer the antagonist in combination with the analgesic to treat at least one of a viral and bacterial cold. Suitable instructions may be provided in various forms, and it should be recognized that such instruction may be in form of a package insert, a package label, an advertisement (e.g., flier, TV/radio ad, etc.), a regulatory filing (e.g., with FDA), or verbally by a physician or pharmacist. Therefore, particularly preferred methods of advertising a health care product include those in which sale of at least one of an H2-histamine receptor antagonist and an anti-pyretic, analgesic agent are advertised in one step. In another step, information is provided (e.g., printed, displayed, or in audible form) that at least one of a viral and bacterial cold can be treated with a combination of the H2-histamine receptor antagonist and the anti-pyretic, analgesic agent.

Suitable methods of providing a health care product will thus include a step in which at least one of (a) an H2-histamine receptor antagonist and (b) acetaminophen or other anti-pyretic, analgesic agent are provided. In another step, an instruction is provided to administer the H2-histamine receptor antagonist in combination with the acetaminophen or other anti-pyretic, analgesic agent to treat at least one of a viral and bacterial cold. Such health care products include most preferably over-the-counter products. In one particularly preferred aspect, an H2-histamine receptor antagonist is offered for sale in a sales display in a position proximal (e.g., within 1-2 meters, or within 2-4, and less preferably within 4-8 meters) to a position in which an analgesic or a cold or flu medicine is offered for sale.

Of course it should be recognized that use of contemplated compounds is not limited to treatment of a viral cold or flu, but that contemplated compositions may be administered for treatment of numerous viral and/or bacterial infections. Typically, at least one of the symptoms associated with the bacterial and/or viral infection is reduced, and it should further be appreciated that besides symptomatic relief, contemplated compositions may also be effective in reduction of bacterial and/or viral titers. While not wishing to be bound by any theory or hypothesis, the inventor contemplate that cimetidine (and selected other H2 antagonists) may block suppressor T-cells and may therefore contribute to an enhanced state of immunity. Furthermore, it should be appreciated that cimetidine (and selected other H2 antagonists) also inhibit hepatic oxidative metabolism by most cytochrome P450 enzymes and, thus, may increase the desired effect of acetaminophen, NSAID, or other anti-pyretic, analgesic agent.

It should be noted that cimetidine was previously reported in treatment of herpes simplex and HIV infections, and that such treatment effectively reduced outbreak and duration of herpes zoster. However, less encouraging results were obtained with HIV patients. In further reported uses for cimetidine, improvements of various immune functions were reported (probably due to blockade of selected suppressor T-cells).

Thus, specific embodiments and applications of compositions and methods for treatment of viral and bacterial infections have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A pharmaceutical kit comprising at least one of an H2-histamine receptor antagonist and an anti-pyretic, analgesic agent, and an instruction to administer the antagonist in combination with the anti-pyretic, analgesic to treat at least one of a viral and bacterial cold.

2. The pharmaceutical kit of claim 1 wherein the H2-histamine receptor antagonist is included in an over-the-counter product.

3. The pharmaceutical kit of claim 1 wherein the H2-histamine receptor antagonist is selected from the group consisting of famotidine, ranitidine hydrochloride, omeprazole, esomeprazole, and cimetidine.

4. The pharmaceutical kit of claim 1 wherein the anti-pyretic, analgesic agent is included in an over-the-counter product.

5. The pharmaceutical kit of claim 1 wherein the anti-pyretic, analgesic agent is selected from the group consisting of acetaminophen, ibuprofen, naproxen, acetyl salicylic acid, celecoxib, and rofecoxib.

6. The pharmaceutical kit of claim 1 comprising the H2-histamine receptor antagonist in solid form for oral administration, and the anti-pyretic, analgesic agent in solid form for oral administration.

7. The pharmaceutical kit of claim 1 wherein the instruction instructs a user to administer the H2-histamine receptor antagonist orally using multiple doses of up to 1500 mg total daily, and wherein the instruction further instructs a user to administer the anti-pyretic, analgesic agent orally using multiple doses of up to 1000 mg total daily.

8. The pharmaceutical kit of claim 1 wherein the instruction instructs a user to orally administer the H2-histamine receptor antagonist together with the anti-pyretic, analgesic agent.

9. A method of providing a health care product, comprising
providing at least one of an H2-histamine receptor antagonist and an anti-pyretic, analgesic agent; and
providing an instruction to administer the H2-histamine receptor antagonist in combination with the anti-pyretic, analgesic agent to treat at least one of a viral and bacterial cold.

10. The method of claim 9 wherein at least one of the H2-histamine receptor antagonist and the anti-pyretic, analgesic agent are included in an over-the-counter product.

11. The method of claim 9 wherein the H2-histamine receptor antagonist is selected from the group consisting of famotidine, ranitidine hydrochloride, omeprazole, esomeprazole, and cimetidine.

12. The method of claim 9 wherein the anti-pyretic, analgesic agent is selected from the group consisting of acetaminophen, ibuprofen, naproxen, acetyl salicylic acid, celecoxib, and rofecoxib.

13. The method of claim 9 wherein the instruction instructs a user to administer the H2-histamine receptor antagonist orally using multiple doses of up to 1500 mg total daily, and wherein the instruction further instructs a user to administer the anti-pyretic, analgesic agent orally using multiple doses of up to 1000 mg total daily.

14. The method of claim 9 wherein the instruction instructs a user to orally administer the H2-histamine receptor antagonist together with the anti-pyretic, analgesic agent.

15. A method of providing an over-the-counter health care product, comprising
providing an H2-histamine receptor antagonist; and
offering the H2-histamine receptor antagonist for sale in a sales display in a position proximal to a position in which an anti-pyretic, analgesic or a cold or flu medicine is offered for sale.

16. The method of claim 15 wherein the H2-histamine receptor antagonist is offered for sale in a position proximal to a position in which the cold or flu medicine is offered for sale.

17. The method of claim 15 wherein the H2-histamine receptor antagonist comprises cimetidine, and the an anti-pyretic, analgesic or a cold or flu medicine includes acetaminophen.

18. A method of advertising a health care product, comprising
advertising sale of at least one of an H2-histamine receptor antagonist and an anti-pyretic, analgesic agent; and
providing an item of information that at least one of a viral and bacterial cold can be treated with a combination of the H2-histamine receptor antagonist and the anti-pyretic, analgesic agent.

19. The method of claim 18 wherein the H2-histamine receptor antagonist is selected from the group consisting of famotidine, ranitidine hydrochloride, omeprazole, esomeprazole, and cimetidine, and wherein the anti-pyretic, analgesic agent is selected from the group consisting of acetaminophen, ibuprofen, naproxen, acetyl salicylic acid, celecoxib, and rofecoxib.

20. The method of claim 18 wherein the information comprises an instruction that instructs a user to administer the H2-histamine receptor antagonist orally using multiple doses of up to 1500 mg total daily, and wherein the instruction further instructs a user to administer the anti-pyretic, analgesic agent orally using multiple doses of up to 1000 mg total daily.
